(19) United States
(12) Patent Application Publication
(10) Pub. No.: US 2010/0057094 A1
(43) Pub. Date: Mar. 4, 2010

(54) IOL Forceps With Shaped Jaws

(76) Inventor: Takayuki Akahoshi, Tokyo (JP)

Correspondence Address: LAW OFFICES OF JERRY A. SCHULMAN, 1S376 SUMMIT AVENUE, COURT C, OAKBROOK TERRACE, IL, 60181, US

(21) Appl. No.: 12/544,783

(22) Filed: Aug. 20, 2009

Related U.S. Application Data
(60) Provisional application No. 61/092,344, filed on Aug. 27, 2008.

(51) Int. Cl. A61F 9/007

(52) U.S. Cl. 606/107

(57) ABSTRACT

A forceps for gripping an intraocular lens (IOL) has first and second jaws moveable toward on another. One jaw is formed with a first curvature and the remaining jaw is formed with a second curvature complementary to the first. When an IOL is gripped between the jaws, the first and second curvatures act to partially curve or fold the IOL, facilitating the insertion of the IOL into an IOL cartridge.

### PRIORITY

This application claims priority from U.S. Patent Application Ser. No. 61/092,344, filed 27 Aug. 2008, which is hereby incorporated herein in its entirety.

### FIELD OF THE INVENTION

The present invention relates generally to instruments for eye surgery and, more particularly, to a forceps having a curved jaw to facilitate the loading of an intraocular lens (IOL) into a lens injection cartridge.

### BACKGROUND OF THE INVENTION

Removing an injured or diseased lens from the eye and replacing it with a foldable, artificial intraocular lens (IOL) is an accepted ophthalmic procedure. The use of hydrogels, silicones and soft acrylics have made replacement IOLs easy to fold, and a number of techniques have been developed to fold the IOL and inject it through an incision smaller than would be required if the IOL were unfolded. IOLs typically have wire-like haptics attached which act to keep the lens from moving once it has been inserted into the eye. The orientation of these haptics is also a concern when an IOL is being inserted.

One such technique is to place the IOL into an injector cartridge and use the plunger of an injector tool to push the IOL from the cartridge and into the capsular bag. A well-known example of such a cartridge is the Alcon “D” cartridge provided by Alcon Laboratories, Inc., described in U.S. Pat. Nos. 5,947,976, 6,083,231 and 6,143,001. The cartridge consists principally of a cartridge body and a cartridge nozzle and a bore extending from an open end of the cartridge body to the open end of the nozzle. The IOL is placed in the cartridge body, the cartridge is placed in an injector tool and a plunger is advanced to push the IOL through the cartridge body and through the nozzle, expressing the IOL into the eye's capsular bag.

When first placed into the Alcon “D” cartridge, the IOL is in an unfolded state. As the IOL is forced through the cartridge body, the asymmetric shape of the passage through which the IOL is pushed causes the IOL to fold or roll into a more compact configuration which allows the IOL to be pushed through the narrowed cartridge nozzle into the eye.

Medical personnel typically use a specially designed forceps to grip the IOL and insert it into the open end of the cartridge body. An example of one such forceps is the model AE 4253 sold by ASICO LLC of Westmont, Ill. Typically, injector forceps have a pair of opposed jaws having flat surfaces which are used to grip the IOL and insert it into the hollow cartridge body. When the IOL has been properly inserted, the cartridge is placed in a cartridge injector and the injector plunger is advanced to push the IOL through the remaining portion of the injector body and through the injector nozzle, into the eye.

Examples of forceps designed to be used with foldable IOLs are well represented in the prior art.

U.S. Pat. No. 5,752,960 (Nallakrishnan) teaches and describes an intraocular lens insertion forceps for inserting an IOL directly into the eye.

U.S. Pat. No. 5,662,659 (McDonald) teaches and describes an endwise adjustable eye forceps designed to allow the placement of an IOL directly into the eye.

U.S. Pat. No. 5,178,622 (Lehner) teaches and describes an instrument for implanting a soft intraocular IOL that includes a pair of jaws which, although curved, are designed to hold the IOL flat therebetween.

U.S. Pat. No. 5,100,410 (Dulebohn) teaches and describes a means and method for facilitating folding of an intraocular IOL that includes the use of a forceps with angle jaws and a forming device used to automatically position and move the jaws in order to fold the IOL.

U.S. Pat. No. 4,844,065 (Faulkner) teaches and describes an intraocular lens inserting tool and method having a pair of jaws that are curved away from each other and are used to compress an IOL into a folded shape when the IOL is held by a second pair of forceps with a pair of straight jaws.

U.S. Pat. No. 4,726,367 (Shoemaker) teaches and describes a surgical instrument for implanting an intraocular IOL having a pair of opposed jaws that slide one toward the other to hold an IOL in between.

I have determined that forming the IOL into a partial fold will facilitate the initial placement of the IOL into the shaped opening at the end of the cartridge. Holding the IOL in a partially curved position also allows the forwardmost or leading haptic of the IOL to be more easily inserted into the cartridge opening.

While the following describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

### BRIEF DESCRIPTION OF THE INVENTION

An IOL-holding forceps has a pair of opposed jaws formed with complementary curves such that the jaws nest when forced together. A foldable IOL placed between the jaws will, when the forceps is closed, be bent into a curved or partially folded position allowing the IOL to be more easily inserted into an injector cartridge. A guide groove is formed on the upper forceps jaw so that the user knows that the forceps are positioned to force the center of the IOL downward and the periphery of the IOL upward to begin the folding process.

### BRIEF DESCRIPTION OF THE DRAWINGS

These and further aspects of the present invention will become more apparent upon consideration of the accompanying drawings in which aspects of the invention are illustrated by way of example and not to scale.

FIG. 1 is a top plan view of a prior art IOL injection cartridge;

FIG. 2 is a view along line 2-2 of FIG. 1;

FIG. 3 is a perspective view of an IOL forceps embodying certain aspects of the present invention;

FIG. 4 is a partial sectional view along 4-4 of FIG. 3;

FIG. 5 is a lateral view of a portion of the jaws of the forceps shown in FIG. 3;

FIG. 6 is a partial top view of FIG. 5;

FIG. 7 is a partial sectional view showing an unfolded IOL positioned in the jaws of FIG. 4;

FIG. 8 is a partial sectional view of the jaws of FIG. 4 showing a partially folded IOL held therein; and

FIG. 9 is a partial top view of a partially folded IOL held in the forceps of FIG. 3.

### DETAILED DESCRIPTION OF THE DRAWINGS

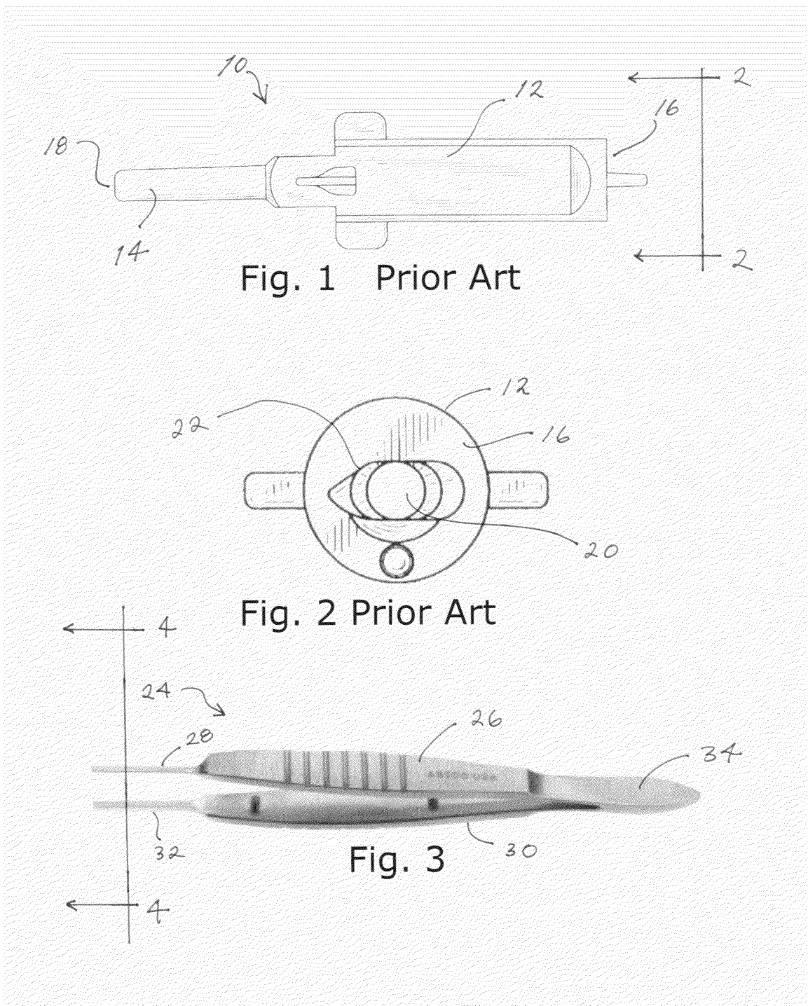

Referring now to FIG. 1, the numeral 10 identifies a prior art IOL cartridge having a hollow body 12 and a nozzle 14. As described above, cartridge 10 has an asymmetric central bore beginning at open cartridge end 16 and terminating at nozzle tip 18. After an unfolded IOL is inserted at open end 16, cartridge 10 is placed in a cartridge injector (not shown) and a plunger is advanced to push the IOL through cartridge 10, with the IOL emerging from nozzle tip 18 in a rolled or folded configuration.

Referring now to FIG. 2, bore 20 is seen as viewed from cartridge end 16 with an IOL intended to be passed through bore 20 inserted through cartridge port 22. As described in U.S. Pat. No. 5,752,960, an IOL inserted into port 22 is oriented such that the larger dimension of the IOL is aligned with the larger dimension of port 22.

Referring now to FIG. 3, an IOL forceps 24 embodying certain aspects of the present invention is shown. Forceps 24 has an upper handle 26 terminating in an upper jaw 28 and a lower handle 30 terminating at a lower jaw 32.

Upper and lower handles 26, 30 are joined at forceps end 34 and are biased such that in an unstressed position, upper jaw 28 and lower jaw 32 are separated. In use, upper handle 26 and lower handle 30 are squeezed together bringing upper jaw 28 into contact with lower jaw 32 to grip an IOL placed between upper and lower jaws 28, 32.

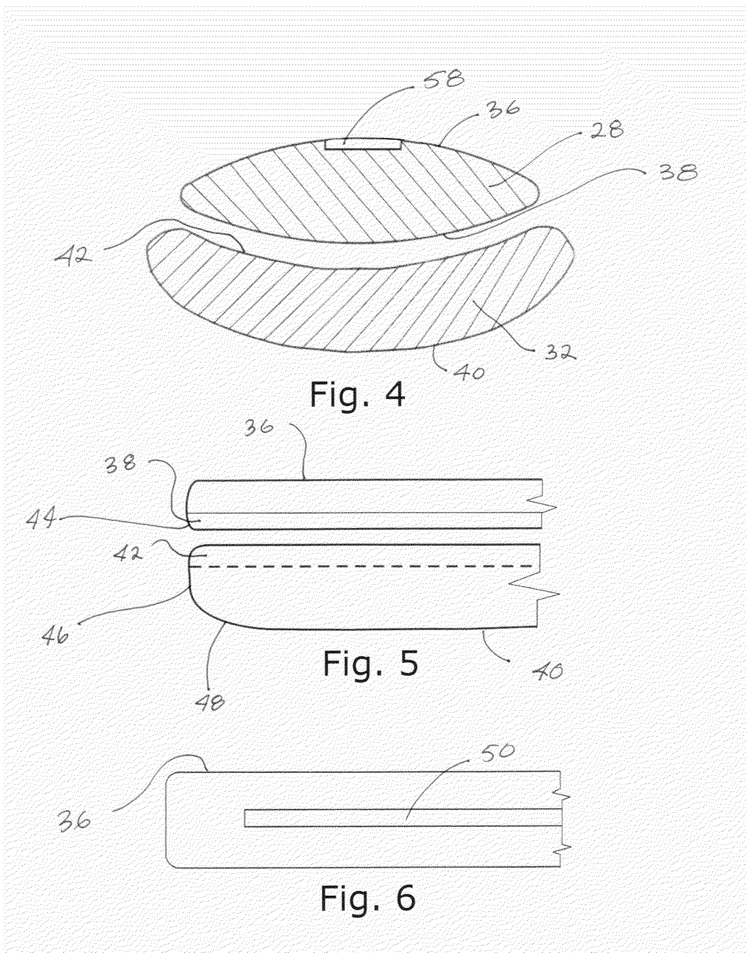

Referring now to FIG. 4, a view along 4-4 of FIG. 3 is shown as a partial sectional view of upper jaw 28 and lower jaw 32. As seen in FIG. 4, upper jaw 28 is formed with an outer surface 36 and an inner surface 38. In the embodiment shown, upper inner surface 38 is formed as a convex curve.

Similarly, lower jaw 32 has an outer surface 40 and an inner surface 42.

As seen, lower inner surface 42 is formed as a concave curve approximating and forming a nesting surface for upper inner surface 38.

It should be noted that in the embodiment shown in FIG. 4, outer surface 36 and lower outer surface 40 are also shown as curved surfaces. Other shapes may also be used and the selected shape may be dictated, inter alia, by the shape of the opening at open end 16 of cartridge 10.

Referring now to FIG. 5, a partial lateral view of upper and lower jaws 36, 40 is shown. Upper inner surface 38 is shown slightly separated from lower inner surface 42.

Upper jaw 36 terminates in an upper jaw tip 44 while lower jaw 40 terminates at a lower jaw tip 46. As seen in FIG. 5, both upper and lower jaw tips 44, 46 are shown as having polished or rounded edges to lessen the possibility of damage to an IOL and to avoid friction with bore 20 of cartridge 10. As an added safety factor, the lowermost forward portion 48 of tip 46 is also rounded and polished.

Referring now to FIG. 6, a partial top view of upper jaw 36 is shown having a guide strip 50 formed therealong to identify it as the upper jaw. In this manner, one using the forceps will know that the visible jaw is either the upper or lower jaw and will then know in what direction an IOL placed between the jaws will be folded. It should be noted that guide strip 50 may be a painted or applied color strip or, as seen in FIG. 4 as a groove 58 formed in outer surface 36 of upper jaw 28, so long as it is visible to the medical personnel using it. It is also contemplated that a similar guide may be formed on the upper surface of upper handle 26.

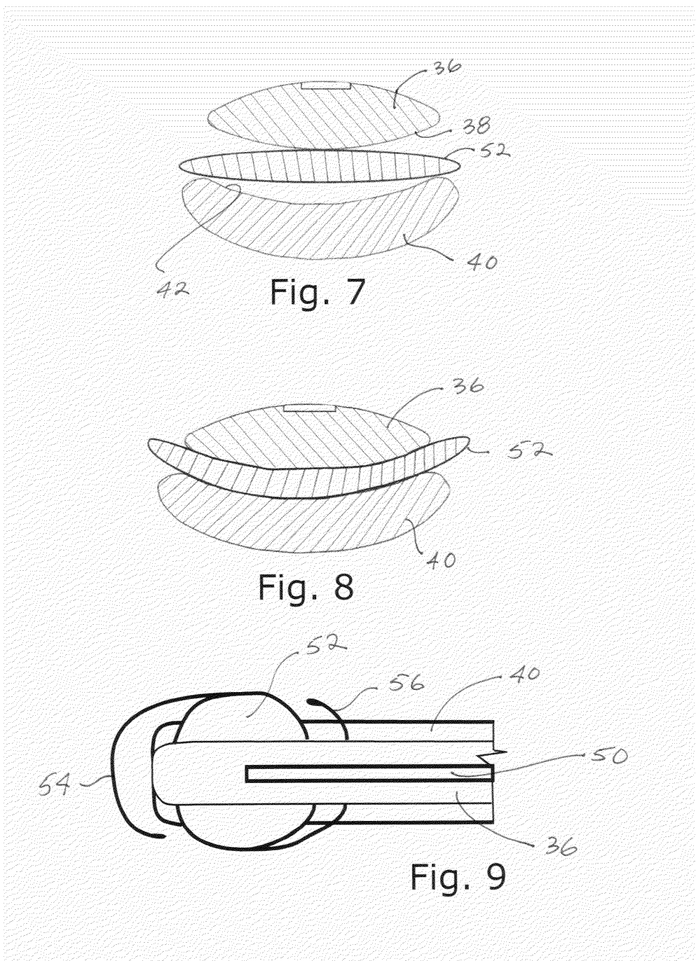

Referring now to FIG. 7, the view of FIG. 4 is shown with an unfolded IOL 52 placed between upper jaw 36 and lower jaw 40. As handles 26, 30 are squeezed together, it can be appreciated that an IOL placed between upper and lower jaws 36, 40 will be partially folded to match the curvatures of upper and lower inner surfaces 38, 42.

As shown in FIG. 8, when handles 26, 30 are squeezed together IOL 52 is partially folded, with the curvature of IOL 52 substantially approximating the shapes of the curves of upper and lower inner surfaces 38, 42.

Referring now to FIG. 9, a partial top view of upper jaw 36 and lower jaw 40 gripping IOL 52 is shown. In the view shown in FIG. 9, IOL 52 has a leading haptic 54 and a trailing haptic 56 with leading haptic 54 oriented to extend forward of IOL 52 as jaws 36, 40 are inserted into cartridge 10.

In use, forceps 24 is held with guide strip 50 facing upward. An IOL 52 is placed between jaws 36, 40 with haptics 54, 56 oriented in a desired position for insertion into cartridge 10. Jaws 36, 40 are then closed, partially folding IOL 52 and holding IOL 52 firmly enough to allow it to be inserted into bore 20 of cartridge 10. Thereafter, jaws 28, 32 are released and forceps 24 is removed from cartridge 10. If desired, forceps 24 can be turned to any desired position to insert IOL 52, with guide strip 50 used to determine the direction in which IOL 52 is partially curved or folded.

Cartridge 10 may thereafter be placed in its operative position on an IOL injector to be used to move IOL 52 down the remaining portion of bore 20 to be subsequently fully folded and expressed through nozzle 14 into the eye.

While surfaces 38, 42 have been described herein as curved, it should be understood that other surface shapes can be used as well, so long as IOL 52 is partially folded when jaws 28, 32 are brought together.

I claim:

1. A forceps for grasping an intraocular lens, said forceps comprising:
a first jaw;
a second jaw,
said first and second jaws opposed one to the other and selectively moveable toward and away from a closed position,
said first and second jaws being shaped to grasp and partially fold said intraocular lens when said first and second jaws are moved toward said closed position.

2. The apparatus as recited in claim 1 wherein said first jaw has an upper surface and a lower surface,
at least a first portion of said first jaw lower surface formed with a first curve of selected shape; and
said second jaw has an upper surface and a lower surface,
at least second portion of said second jaw upper surface formed with a second curve of selected shape,
said first and second curved portions shaped to substantially nest when said first and second jaws are moved toward said closed position.

3. The apparatus as recited in claim 1 wherein said first jaw terminates at a first jaw tip and said second jaw terminates at a second jaw tip,
at least a portion of one said jaw tip polished to a smoothness sufficient to decrease the friction between said jaw tip portion and a selected surface.

4. The apparatus as recited in claim 2 wherein said first jaw has indicia formed on a portion of said upper surface to identify said first jaw.

5. The apparatus as recited in claim 2 wherein said first curve is convex and said second curve is concave.
